(19) United States
(12) Patent Application Publication
(10) Pub. No.: US 2016/0060288 A1
(43) Pub. Date: Mar. 3, 2016

(54) Ester derivatives of 7-alpha-[9-(4,4,5,5,5-pentafluoropentylsulfinyl)nonyl)-estra-1,3,5(10)-triene-3,17beta-diol having anticancer activity and preparation method thereof

(71) Applicant: XI'ANLIBANG PHARMACEUTICAL TECHNOLOGY CO., LTD., Xi'an, SX (CN)

(72) Inventors: Yaqi Jiao, Xi'an (CN); Jiucheng Wang, Xi'an (CN); Renle Hu, Xi'an (CN)

(21) Appl. No.: 14/781,650

(22) PCT Filed: Apr. 18, 2013
(86) PCT No.: PCT/CN2013/074363

§ 371 Date: Oct. 1, 2015

(51) Int. Cl. C07J 1/00; A61K 9/00

(52) U.S. Cl. CPC C07J 1/0074; C07J 1/007; A61K 9/0019

(57) ABSTRACT

The present invention provides a class of fulvestrant ester derivatives and preparation method thereof. Such a compound is an aliphatic ester formed by esterifying the —OH at positions C-3 and C-17 of fulvestrant, having a structure of the following formula:

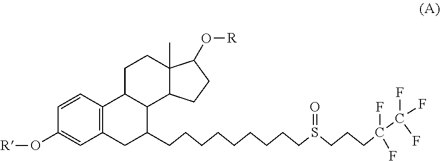

In the formula, substituent R is H, alkanoyl or alkenoyl having 2 to 22 carbon atoms or stereoisomers thereof; substituent R′ can be H, alkanoyl or alkenoyl having 2 to 4 carbon atoms or stereoisomers thereof; such an aliphatic ester compound being used as a pro-drug can improve the stability of a compound, meanwhile the decrease of polarity can enable them to be easily made into preparations like lipid emulsion, microsphere etc., and avoid the degradation of the compound caused by factors like high temperature etc. and the use of an organic solution during preparation process.

### TECHNICAL FIELD

The invention belongs to the field of medicine, specifically relates to a preparation method of compounds of general Formula A, and more specifically relates to the ester derivatives of 7-a-[9-(4,4,5,5,5-pentafluoropentylsulfinyl)nonyl]-estra-1,3,5(10)-triene-3,17β-diol and preparation method thereof.

### BACKGROUND ART

7-α-[9-(4,4,5,5,5-pentafluoropentylsulfinyl)nonyl]-estra-1,3,5(10)-triene-3,17β-diol, also referred to as fulvestrant, having a general Formula B, is a novel estrogen receptor blocking agent in the treatment of postmenopausal advanced breast cancer which fails to respond to anti-estrogen therapy and which is estrogen receptor positive.

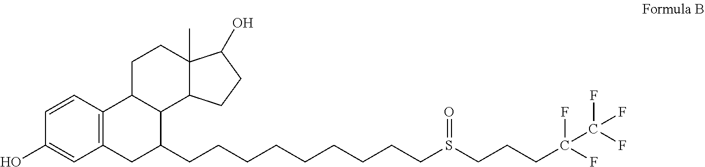

The most important feature of breast cancer is that its occurrence and development associates with the estrogen level and metabolism thereof in vivo. Studies have shown that estrogen receptor (ER) was found in tumor cells of many patients with breast cancer, and tumor growth was stimulated by estrogen. Thus, one of the main methods for treating breast cancer is reducing the concentration of estrogen or blocking the binding of estrogen to its receptor to inhibit the growth and reproduction of tumor cells. Fulvestrant can competitively binding to estrogen receptors, with affinity similar to estradiol; it can also block the receptors, inhibit the binding of estrogen, stimulate deformation of receptors, and reduce ER concentration to destroy tumor cells. Fulvestrant can down-regulate ER protein in human breast cancer cells, down-regulate ER in tumor cells, and minimize tumor growth. Since fulvestrant does not change the condition of existing tumor ER and does not affect the generation of new ER, the tumor continues to be “programmed” as ER positive. In this way, fulvestrant continues to have therapeutical effects. Its greatest advantage is that it does not have partial agonistic action and estrogen-like activity of common antiestrogen drugs.

Currently, many commercial available preparations of fulvestrant use oil as an excipient for the following two reasons. On the one hand, fulvestrant, which is of poor stability and easy to degrade, is generally stored at −20, and should not be stored at room temperature for too long, otherwise its purity would be affected. Although the mechanism of its degradation is not clear, it is generally believed that the main reason for affecting its stability lies in the presence of —OH at positions C-3 and C-17. Meanwhile, the presence of —OH at 3- and 17-positions increases the polarity of drugs and the stimulation of drugs on the gastrointestinal tract, thus it can only be prepared into injection.

On the other hand, like other steroids, fulvestrant, which is difficult to be formulated due to certain physical properties, is a molecule with high lipophilicity and extremely low water solubility of about 10 ng/mL. Its solubility is provided in U.S. Pat. No. 5,183,514 and CN1394141A (mg/mL, 25° C.) (water 0.001, peanut oil 0.45, sesame oil 0.58, castor oil 20, Migloyl 810 3.06, Migloyl 812 2.72, ethyl oleate 1.25, benzyl benzoate 6.15, isopropyl myristate 0.80, Span 85 3.79, ethanol>200, benzyl alcohol>200). It can be seen that, even in castor oil with the maximum solubility, it is impossible to provide a concentration of fulvestrant that meets clinical requirement for administration. Therefore, many fulvestrant preparations in the marketplace not only use oil as solvent, but also add other excipients, such as ethanol, benzyl benzoate, benzyl alcohol and the like, to facilitate solubilizing. In this way, it can be formulated into intramuscularly injectable injections with content not less than 45 mg/mL, which can maintain effective plasma concentration (2.5 ng/mL) for 2 weeks. However, the addition of such solvents may increase the risk of precipitation of the drug in the preparations and cause irritation at injection sites.

Thus it can be seen that, the problem to be solved in prior art is how to make structural improvements to fulvestrant, especially to —OH at positions C-3 and C-17, so as to reduce irritation to human body and increase its lipophilicity, thereby making it more easily to be formulated into preparations for human use, while maintain its inhibition effect on cancer cell.

### SUMMARY OF INVENTION

Therefore, one object of the present invention is to make improvement to —OH at C-3 and C-17 positions of fulvestrant structure, and esterify fulvestrant into ester (including carboxyl carbon) compounds having 2 to 22 carbon atoms at C-17 position and ester (including carboxyl carbon) compounds having 2 to 4 carbon atoms at C-3 position, so as to increase the drug stability and its solubility in lipophilic solvents.

The object of the present invention is achieved by the following technical solutions:

The present invention provides a compound of Formula A:

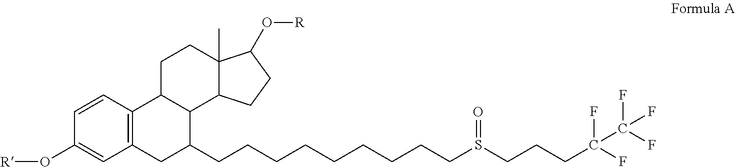

wherein:

substituent R′ is selected from H, alkanoyl or alkenoyl having 2 to 4 carbon atoms,

substituent R is selected from H, alkanoyl or alkenoyl having 2 to 22 carbon atoms;

preferably,

substituent R′ is H, and substituent R is selected from alkanoyl or alkenoyl having 11 to 22 carbon atoms;

preferably,

said substituent R is selected from alkanoyl having 11 to 22 carbon atoms, preferably undecanoyl, hexadecanoyl, docosanoyl or 2-[(3′,3′)-dimethyl-1′-methyl]butyl-5-methyl-(7,7)-dimethyl-octanoyl;

preferably,

said substituent R is selected from alkenoyl containing 1 to 6 carbon-carbon double bonds and having 11 to 22 carbon atoms, wherein said carbon-carbon double bonds can either be distributed in the main chain, or in the branched chain;
preferably,

said substituent R is selected from undec-2-enoyl, eicosa-5,8,11,14,17-pentaenoyl and docosa-(4,7,10,13,16,19)-hexaenoyl;

preferably,

when substituent R′ is selected from alkanoyl having 2 to 4 carbon atoms, said alkanoyl is acetyl or butyryl;

preferably,

said substituent R is selected from alkanoyl or alkenoyl having 11 to 22 carbon atoms, preferably 2-[(3′,3′)-dimethyl-1′-methyl]butyl-5-methyl-(7,7)-dimethyl-octanoyl or undec-2-enoyl.

Exemplarily, said compounds may have structures of the following formulae. The structural formulae of fulvestrant esters I-XI are shown below:

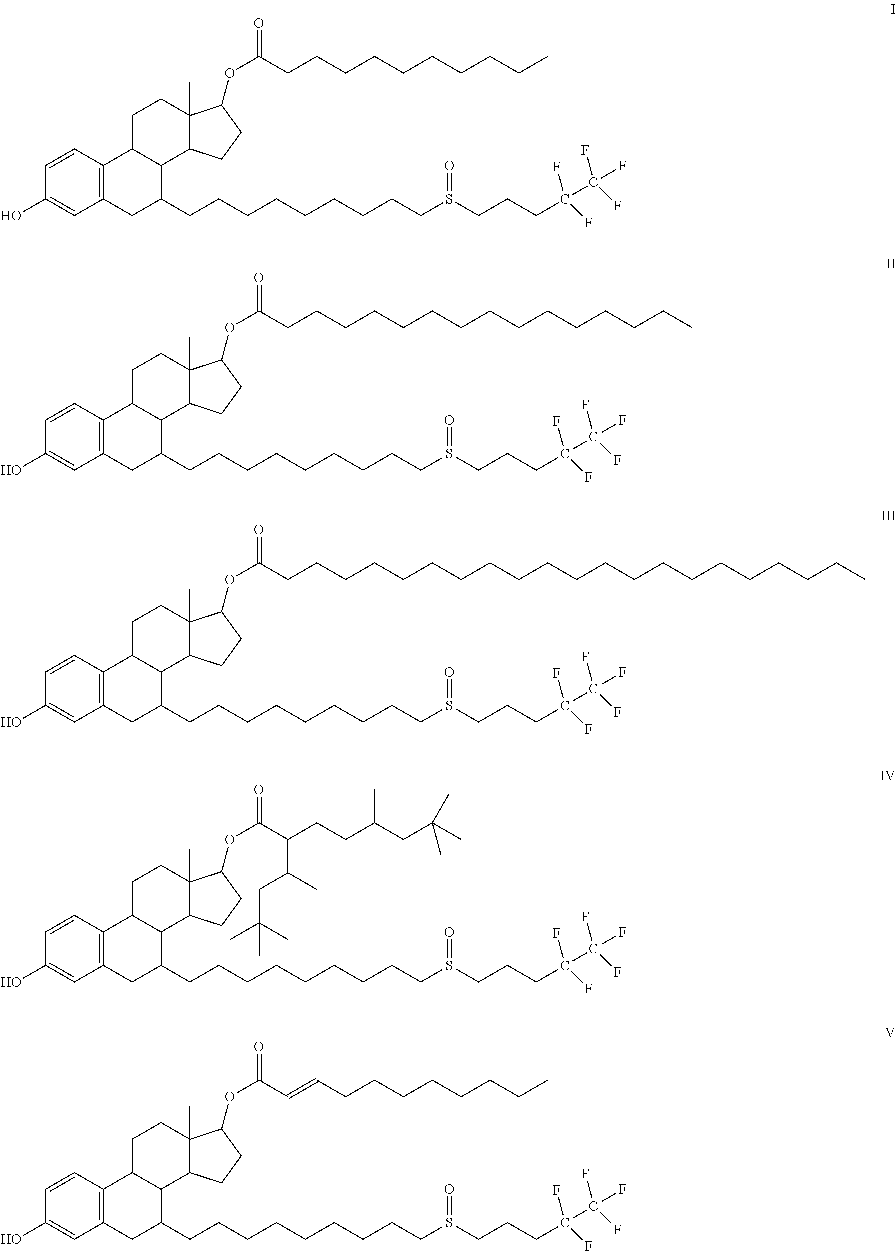 [SMILES: CCCCCCCCCCC(=O)OC1CCC2C3C(CCCCCCCCCS(=O)CCCC(F)(F)C(F)(F)F)Cc4cc(O)ccc4C3CCC12C ; CCCCCCCCCCCCCCCC(=O)OC1CCC2C3C(CCCCCCCCCS(=O)CCCC(F)(F)C(F)(F)F)Cc4cc(O)ccc4C3CCC12C ; CCCCCCCCCCCCCCCCCCCCCC(=O)OC1CCC2C3C(CCCCCCCCCS(=O)CCCC(F)(F)C(F)(F)F)Cc4cc(O)ccc4C3CCC12C ; CC(CCC(C(=O)OC1CCC2C3C(CCCCCCCCCS(=O)CCCC(F)(F)C(F)(F)F)Cc4cc(O)ccc4C3CCC12C)C(C)CC(C)(C)C)CC(C)(C)C ; CCCCCCCC/C=C/C(=O)OC1CCC2C3C(CCCCCCCCCS(=O)CCCC(F)(F)C(F)(F)F)Cc4cc(O)ccc4C3CCC12C]

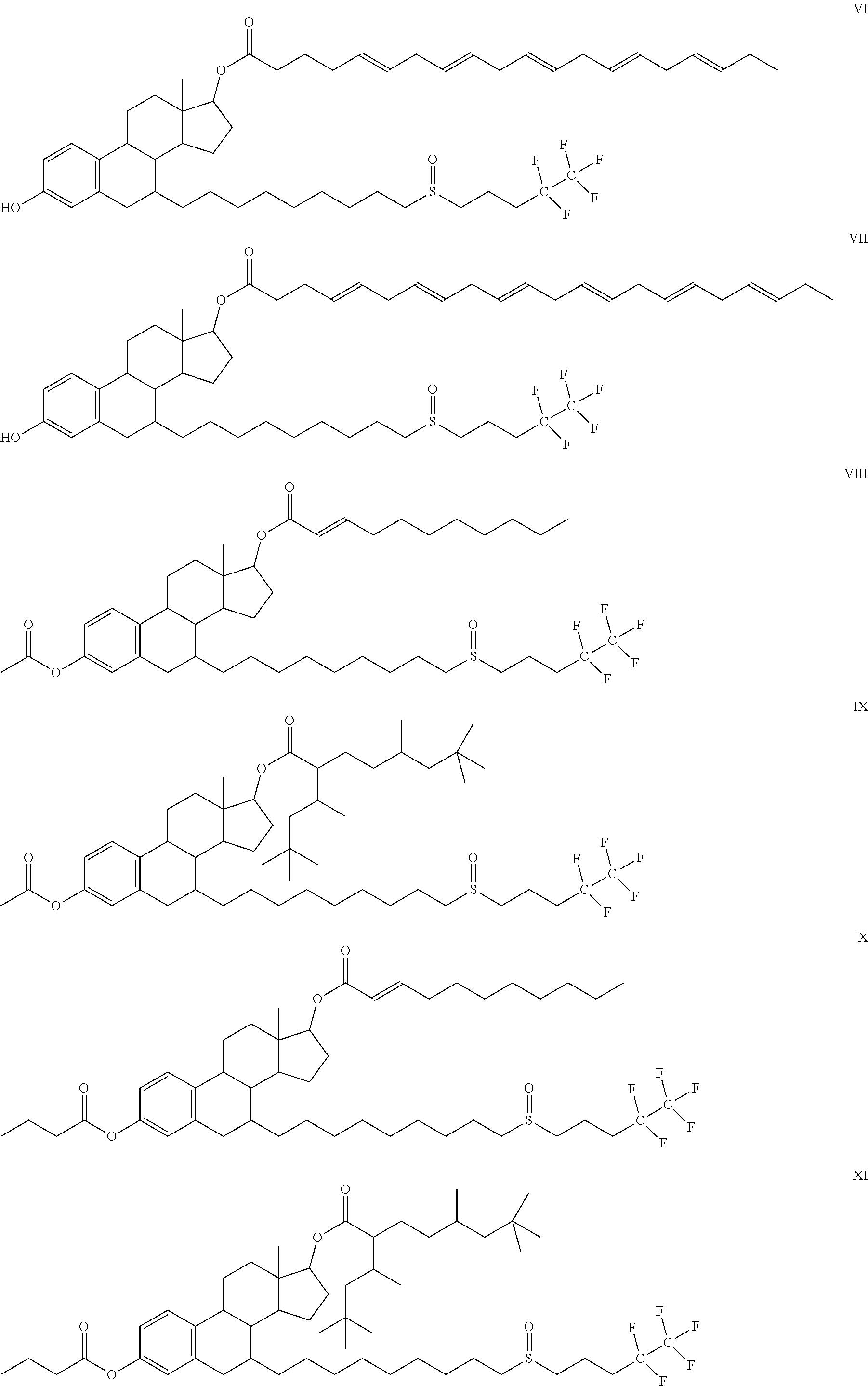

Furthermore, the present invention provides a process for preparing the compounds described as above, said process comprises the steps of:

a) acylating the —OH at C-17 position of compound of formula B: a compound of formula B is mixed with an alkaline reagent, an organic acid and a catalyst in a solvent at room temperature under stirring to form a reaction mixture, said reaction mixture is reacted to obtain a crude product of compound of Formula A with C-17 position acylated;

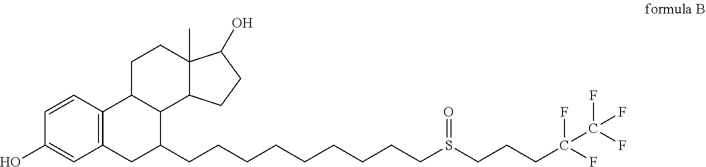 [SMILES: CC12CCC3c4ccc(O)cc4CC(CCCCCCCCCS(=O)CCCC(F)(F)C(F)(F)F)C3C1CCC2O]

b) purifying the crude product obtained in step a) to remove the by-product N,N-dicycloalkylurea and obtain a purified product of compound of Formula A with C-17 position acylated;

when said substituent R′ in the compounds is not H, said process further comprises the steps of:

c) acylating C-3 position of the purified product with C-17 position acylated obtained in step b): the purified product with C-17 position acylated obtained in step b) is mixed with an alkaline reagent, an organic acid and a catalyst in a solvent at room temperature under stirring to be reacted to obtain a crude product of compound of Formula A with C-17 and C-3 positions acylated;

d) purifying the crude product obtained in step c) to obtain a purified product of compound of Formula A.

Wherein, in step a), said alkaline reagent is selected from pyridine, 2-methylpyridine, 3-methylpyridine, 4-methylpyridine, 2-ethylpyridine, 3-ethylpyridine, 4-ethylpyridine, 5-ethylpyridine, 2-methyl-5-ethylpyridine, 2-dimethylaminopyridine, 4-dimethylaminopyridine, preferably 4-dimethylaminopyridine; said solvent is selected from methyl chloride, methylene chloride, chloroform; said catalyst is dehydrating agent, preferably N,N-dicyclohexylcarbodiimide; said organic acid is alkyl acid or alkenyl acid having 2 to 22 carbon atoms; in step b), said purifying comprises the step of dissolving the crude product obtained in step a) in tetrahydrofuran or ethyl acetate to form a solution, then settling the solution with n-hexane or mixed solvent of n-hexane-ethyl acetate, separating and purifying the settled solution by silica-gel column chromatography and/or neutral alumina adsorption; in step c), said alkaline reagent is selected from pyridine, 2-methylpyridine, 3-methylpyridine, 4-methylpyridine, 2-ethylpyridine, 3-ethylpyridine, 4-ethylpyridine, 5-ethylpyridine, 2-methyl-5-ethylpyridine, 2-dimethylaminopyridine, 4-dimethylaminopyridine, preferably 4-dimethylaminopyridine; said solvent is selected from tetrahydrofuran, ethyl acetate, preferably tetrahydrofuran; said catalyst is dehydrating agent, preferably N,N-dicyclohexylcarbodiimide; said organic acid is alkyl acid or alkenyl acid having 2 to 4 carbon atoms; in step d), said purifying is carried out by silica-gel column chromatography and ethanol elution, wherein mixed solvent of n-hexane-ethyl acetate is used for gradient elution in said silica-gel column chromatography, the volume ratio of n-hexane to ethyl acetate is 50:1-1:1, preferably 40:1/10:1/5:1 for gradient elution.

Furthermore, the present invention provides a composition comprising a compound of Formula A described as above, wherein, said composition is an oiling agent, a fatty agent or a microsphere agent.

Furthermore, the present invention also provides the use of a compound of Formula A described as above or a composition comprising a compound of Formula A for the manufacture of a medicament in the treatment of cancer; said medicament is preferably used to inhibit cancer cells with estrogen receptors, particularly preferably used to inhibit breast cancer cells.

The present invention also provides a method for treating cancer, wherein said method comprises administering to a subject in need a therapeutically effective amount of a compound of Formula A described as above; said method is preferably used to inhibit cancer cells with estrogen receptors, particularly preferably used to inhibit breast cancer cells;

preferably, said compound of Formula A is administered by injection.

Exemplarily, after being formulated into oiling agent, the compound(s) according to the present invention is administered to nude mice bearing human breast cancer MCF-7 tumor by subcutaneous injection to study the tumor inhibition rate. The result showed that such derivatives have anticancer activity for treating breast cancer.

### DESCRIPTION OF THE PREFERRED EMBODIMENTS

Hereinafter, the present invention will be further described in detail in combination with specific embodiments. The examples given are only for illustration, but not for limiting the scope of the present invention.

### Synthesis Examples

Although the alkaline reagent in the examples below is 4-dimethylaminopyridine, it is understood that agents such as pyridine, 2-methylpyridine, 3-methylpyridine, 4-methylpyridine, 2-ethylpyridine, 3-ethylpyridine, 4-ethylpyridine, 5-ethylpyridine, 2-methyl-5-ethylpyridine, 2-dimethylaminopyridine and the like can also be used in the examples below as alkaline reagents.

### Example 1

### Synthesis and Structure Confirmation of Compound II

1) Reaction Treatment

5 g (8.25 mmol) fulvestrant was added into a 500 mL three-necked round-bottom flask and dissolved with 300 mL methylene chloride while stirring. Then, 0.137 g (1.1 mmol) 4-dimethylaminopyridine (DMAP), 2.155 g (8.41 mmol) palmitic acid and 1.672 g (8.23 mmol) N,N-dicyclohexylcarbodiimide (DCC) was added sequentially into said flask. After reacting at room temperature (e.g., 20±5) for 48 h, the reaction was stopped.

2) Post Process

The reaction mixture was filtered to remove precipitated by-product N,N′-dicyclohexylurea (DCU). The filtrate was washed with saturated sodium bicarbonate solution, then washed with water to neutral, and then evaporated by rotary evaporator to remove methylene chloride. Colorless and clear colloidal liquid (8.8 g) was obtained, which was dissolved in an appropriate amount of ethyl acetate, froze in a refrigerator (e.g., the freezing temperature may be −15±3). A small amount of white solid precipitated was washed out and removed by filtration for 3 times. Then, the filtrate was evaporated by rotary evaporator to remove ethyl acetate, and colorless and clear colloidal liquid was obtained. The colorless and clear colloidal liquid was dissolved in a small amount of tetrahydrofuran, then the solution was added to n-hexane to form a large quantity of white solid, which was left to stand and filtrated; the filter cake was dissolved in the aforesaid tetrahydrofuran and settled in n-hexane for 3 times to give white powder product, which was pure Compound II. The product was dried in vacuum at 60 to give 1.5 g II (purity 99.88% as determined by HPLC, C18 column, mobile phase: 67% THF in water, flow rate: 1.0 mL/min, detection wavelength: 220 nm), and the molar yield was 22%.

IR (cm⁻¹): 3209, 2922, 2852, 1607, 1503, 1446, 1385, 1106, 1055, 1014, 982.

¹HNMR (500 MHz, CDCl3, ppm): 0.78 (s, 3H), 0.88 (t, 3H), 1.01-1.52 (t, 32H), 1.59-1.63 (t, 6H), 1.70-1.76 (t, 6H), 1.89-1.94 (t, 2H), 2.10-2.32 (t, 10H), 2.61-2.85 (t, 8H), 3.74 (t, 2H), 6.20 (d, j=10 Hz, 1H), 6.56-7.14 (t, 3H).

¹³CNMR (125 MHz, CDCl3, ppm): 172.67, 154.23, 136.88, 131.04, 126.93, 117.67, 113.01, 82.02, 52.41, 50.83, 46.49, 43.40, 42.05, 38.23, 36.92, 34.74, 34.65, 33.35, 33.24, 31.93, 30.51, 29.92-28.22, 27.24, 25.62, 25.00, 22.63, 14.65, 14.09, 11.12.

### Example 2

### Synthesis and Structure Confirmation of Compound I

1) Reaction Treatment

3 g (4.95 mmol) fulvestrant was added into a 250 mL round-bottom flask and dissolved with 160 mL methylene chloride while stirring. Then 0.0822 g (0.66 mmol) DMAP, 0.96 g (5.05 mmol) undecanoic acid and 1.02 g (4.98 mmol) DCC was added sequentially into said flask. After reacting under stirring at room temperature (e.g., 20±5) for 48 h, the reaction was stopped.

2) Post Process

The reaction system was first frozen to precipitate as much reaction by-product DCU as possible. After being filtered to remove solid DCU, the filtrate was washed with saturated sodium bicarbonate solution, then washed with water to neutral and evaporated by rotary evaporator to remove methylene chloride, to give colorless and clear colloidal liquid, which was dissolved in a small amount of ethyl acetate and then froze in a refrigerator (e.g., the freezing temperature may be −15±3° C.) until no white solid DCU precipitated out. The filtrate was concentrated to remove ethyl acetate, recrystallized from mixed solvent of n-hexane-ethyl acetate, and then filtered to remove white solid precipitated out (unreacted raw material fulvestrant). The mother liquor was spin-dried to give colorless oily matter. Said oily matter was further purified by silica-gel column chromatography (the eluent was n-hexane-ethyl acetate (1:1, volume ratio)) and was then evaporated by rotary evaporator to give 1.0611 g colorless oily matter, which was Compound I (purity 99.104% as determined by HPLC according to the same determination method in Example 1), and the molar yield was 27.7%.

IR (cm-1): 3385, 2926, 2855, 1756, 1494, 1463, 1199, 1152, 1059, 1017, 985, 721.

¹HNMR (500 MHz, CDCl3, ppm): δ 7.28 (s, 1H), 6.83 (d, 1H), 6.77 (d, 1H), 3.73 (t, 1H, J=8 Hz), 2.88-1.17 (t, 57H), 0.89 (s, 3H), 0.77 (s, 3H).

¹³CNMR (125 MHz, CDCl3, ppm): δ 172.64, 148.52, 137.13, 126.91, 122.37, 120.10, 118.64, 81.93, 52.75, 51.03, 46.47, 43.33, 41.67, 38.23, 36.89, 34.50, 34.45, 33.85, 31.89, 29.67, 29.50, 29.63, 29.55, 29.49, 29.46, 29.34, 29.30, 29.26, 29.16, 29.12, 28.80, 28.23, 27.11, 25.70, 25.01, 24.88, 22.66, 14.62, 14.50, 11.50.

### Example 3

### Synthesis and Structure Confirmation of Compound III

3 g (4.95 mmol) fulvestrant was added into a 250 mL round-bottom flask and then dissolved with 160 mL methylene chloride while stirring. Then, 0.0822 g (0.66 mmol) DMAP, 1.87 g (5.05 mmol) docosanoic acid and 1.02 g (4.98 mmol) DCC was added sequentially into said flask. After reacting under stirring at room temperature (e.g., 20±5° C.) for 48 h, the reaction was stopped.

Reaction liquid was treated according to the post process in Example 2 to give 1.016 g white solid powder (purity 92.634%, determined by HPLC) (C18 column, mobile phase: 75% THF in water, flow rate: 1.0 mL/min, detection wavelength: 220 nm), which was Compound III, and the molar yield was 22.1%.

IR (cm-1): 3607, 3424, 2919, 2851, 1754, 1495, 1471, 1199, 1153, 1141, 1112, 1081, 985, 719.

¹HNMR (500 MHz, CDCl3, ppm): δ 7.28 (d, 1H), 6.83 (d, 1H), 6.77 (d, 1H), 3.74 (t, 1H, J=8 Hz), 2.91-1.05 (t, 79H), 0.89 (t, 3H), 0.77 (s, 3H).

¹³CNMR (125 MHz, CDCl3, ppm): δ 172.64, 148.53, 137.13, 126.91, 122.37, 118.64, 81.93, 52.83, 51.11, 46.48, 43.34, 41.68, 38.24, 36.89, 34.50, 33.15, 31.94, 30.56, 29.94, 29.86, 29.71, 29.67, 29.63, 29.62, 29.51, 29.48, 29.37, 29.35, 29.27, 29.17, 29.13, 28.81, 28.23, 27.12, 25.70, 25.01, 24.88, 23.16, 22.66, 14.50, 14.01, 11.50.

### Example 4

### Synthesis and Structure Confirmation of Compound IV

3 g (4.95 mmol) fulvestrant was added into a 250 mL round-bottom flask and then dissolved with 160 mL methylene chloride while stirring. Then, 0.0822 g (0.66 mmol) DMAP, 1.44 g (5.05 mmol) isostearic acid and 1.02 g (4.98 mmol) DCC was added sequentially into said flask. After reacting under stirring at room temperature (e.g., 20±5° C.) for 48 h, the reaction was stopped.

Reaction liquid was treated according to the post process in Example 2 to give 1.0028 g colorless colloid (purity 99.312%, determined by HPLC) (according to the same determination method in Example 3), which was Compound IV, and the molar yield was 23.2%.

IR (cm-1): 3396, 2928, 2866, 1748, 1494, 1466, 1364, 1198, 1149, 1121, 1058, 1017, 984, 720.

¹HNMR (500 MHz, CDCl3, ppm): δ 7.28 (s, 1H), 6.83 (d, 1H), 6.76 (s, 1H), 3.74 (t, 1H, J=8 Hz), 2.35-1.03 (t, 71H), 1.09-0.94 (t, 3H), 0.89 (s, 3H), 0.77 (s, 3H).

¹³CNMR (125 MHz, CDCl3, ppm): δ 171.15, 148.60, 137.03, 126.88, 122.45, 118.72, 81.94, 53.34, 53.04, 52.82, 51.39, 50.96, 48.46, 48.39, 48.32, 46.48, 43.33, 41.68, 38.21, 37.92, 37.86, 37.79, 36.89, 34.50, 33.16, 32.37, 32.05, 31.11, 30.56, 30.06, 29.96, 29.87, 29.69, 29.55, 29.50, 29.37, 29.32, 29.18, 28.81, 28.26, 27.11, 26.09, 25.65, 24.81, 22.66, 21.21, 19.40, 14.61, 14.50, 11.51.

### Example 5

### Synthesis and Structure Confirmation of Compound V

0.36 g (0.6 mmol) fulvestrant was added into a 50 mL round-bottom flask and then dissolved with 25 mL methylene chloride while stirring. Then, 9.93 mg (0.08 mmol) DMAP, 0.113 g (0.61 mmol) undecenoic acid and 0.13 g (0.64 mmol) DCC was added sequentially into said flask. After reacting under stirring at room temperature (e.g., 20±5° C.) for 48 h, the reaction was stopped.

Reaction liquid was treated according to the post process in Example 2 to give light yellow oily matter, which was further purified by silica-gel column chromatography for 3 times and neutral alumina for once and was evaporated to dryness to give 0.1 g light yellow oily matter (purity 96.010%, determined by HPLC) (according to the same determination method in Example 3). The obtained light yellow oily matter was Compound V, and the molar yield was 21.5%.

IR (KBr, cm-1): 3387, 2927, 2855, 1736, 1652, 1494, 1461, 1356, 1312, 1198, 1154, 1121, 1059, 1016, 983, 721.

¹HNMR (500 MHz, CDCl3, ppm): δ 7.27 (t, 1H), 7.15 (t, 1H), 6.87 (s, 1H), 6.82 (s, 1H), 6.43 (t, 2H), 5.99 (t, 1H), 3.74 (t, 1H, J=8 Hz), 3.2-1.1 (t, 51H), 0.89 (t, 3H, J=7 Hz), 0.77 (s, 3H).

¹³CNMR (125 MHz, CDCl3, ppm): δ 170.90, 165.38, 151.71, 148.55, 137.10, 135.55, 126.91, 122.44, 120.94, 120.12, 118.79, 81.93, 52.77, 51.04, 46.50, 43.35, 41.71, 38.27, 36.91, 34.51, 33.18, 31.85, 30.56, 29.94, 29.87, 29.70, 29.62, 29.51, 29.36, 29.34, 29.19, 29.16, 29.09, 28.96, 28.81, 28.23, 27.13, 25.70, 24.88, 22.66, 14.50, 13.50, 11.10.

### Example 6

### Synthesis and Structure Confirmation of Compound VI

0.36 g (0.6 mmol) fulvestrant was added into a 50 mL round-bottom flask and then, dissolved with 25 mL methylene chloride while stirring. Then, 9.93 mg (0.08 mmol) DMAP, 0.185 g (0.61 mmol) eicosapentaenoic acid and 0.13 g (0.64 mmol) DCC was added sequentially into said flask. After reacting under stirring at room temperature (e.g., 20±5° C.) for 48 h, the reaction was stopped.

Reaction liquid was treated according to the post process in Example 2 to give 0.31 mg light yellow oily matter (purity 99.195%, determined by HPLC with the method referred to the method in Example 3), which was Compound VI, and the yield was 58%.

IR (cm-1): 3396, 3012, 2927, 2855, 1756, 1609, 1494, 1456, 1312, 1198, 1137, 1058, 1018, 985, 719.

¹HNMR (500 MHz, CDCl3, ppm): δ 7.28 (t, 1H), 6.84 (t, 1H, J=7.5 Hz), 6.77 (d, 1H), 5.43-5.32 (t, 10H), 3.74 (t, 1H, J=8 Hz), 2.87-1.18 (t, 55H), 0.97 (t, 3H, J=7.5 Hz), 0.77 (s, 3H).

¹³CNMR (125 MHz, CDCl3, ppm): δ 172.38, 137.19, 132.05, 129.07, 128.84, 128.59, 128.29, 128.22, 128.10, 127.89, 127.03, 126.94, 122.34, 118.62, 81.94, 52.76, 51.05, 46.48, 43.34, 41.67, 38.23, 36.89, 34.50, 33.78, 33.15, 30.56, 29.95, 29.86, 29.68, 29.65, 29.50, 29.36, 29.18, 28.82, 28.24, 27.12, 26.56, 25.70, 25.66, 25.65, 25.56, 24.82, 22.66, 20.85, 14.50, 13.50, 11.50.

### Example 7

### Synthesis and Structure Confirmation of Compound VII

0.36 g (0.6 mmol) fulvestrant was added into a 50 mL round-bottom flask and then dissolved with 25 mL methylene chloride while stirring. Then, 9.93 mg (0.08 mmol) DMAP, 0.2 g (0.61 mmol) docosahexenoic acid and 0.13 g (0.64 mmol) DCC was added sequentially into said flask. After reacting under stirring at room temperature (e.g., 20±5° C.) for 48 h, the reaction was stopped.

Reaction liquid was treated according to the post process in Example 2 to give 0.1165 g light yellow oily matter (purity 99.051%, determined by HPLC with the method referred to the method in Example 3), which was Compound VII, and the yield was 21.1%.

IR (cm-1): 3396, 3013, 2927, 2855, 1756, 1609, 1494, 1456, 1358, 1198, 1138, 1059, 1018, 984, 719.

¹HNMR (500 MHz, CDCl3, ppm): δ 7.27 (t, 1H), 6.83 (d, 1H), 6.77 (t, 1H), 5.4-5.3 (t, 12H), 3.74 (t, J=8 Hz, 1H), 2.8-1.1 (t, 55H), 0.97 (t, 3H), 0.77 (s, 3H).

¹³CNMR (125 MHz, CDCl3, ppm): δ 171.88, 148.47, 137.22, 132.05, 129.62, 128.58, 128.33, 128.29, 128.26, 128.11, 128.09, 128.04, 127.89, 127.64, 127.03, 126.93, 122.35, 118.62, 81.94, 52.76, 51.05, 46.48, 43.34, 41.67, 38.23, 36.89, 34.50, 34.34, 33.14, 30.56, 29.94, 29.85, 29.68, 29.65, 29.50, 29.36, 29.18, 28.82, 28.24, 27.12, 25.70, 25.66, 25.64, 25.55, 22.85, 22.66, 22.58, 20.57, 14.30, 14.10, 11.50.

### Example 8

### Synthesis and Structure Confirmation of Compound VIII

1) Reaction Treatment

0.31 g (0.4 mmol) Compound V (synthesized in Example 5), 4 mL (40 mmol) acetic anhydride, 0.2 g (1.6 mmol) 4-dimethylaminopyridine (DMAP) was sequentially added into a 50 mL round-bottom flask. After reflux reacting for 48 h, the reaction was stopped.

2) Post Process

After the reaction system was cooled, it was washed with water to neutral and phase separated. The organic layer was spin-dried and purified by silica-gel column chromatography through gradient eluting (the eluent was n-hexane-ethyl acetate (40:1/10:1/5:1, volume ratio)). Then, the eluent was evaporated to dryness to give milky white colloidal liquid, which was Compound VIII.

IR (cm-1): 3449, 2927, 2855, 1736, 1651, 1494, 1461, 1373, 1360, 1311, 1245, 1198, 1154, 1121, 1045, 1027, 983, 896, 822, 720.

¹HNMR (500 MHz, CDCl3, ppm): δ 7.27 (t, 1H), 7.15 (t, 1H), 6.87 (t, 1 H), 6.81 (d, 1H), 6.40 (t, 1H), 6.00 (d, 1H), 5.63 (t, 1H), 4.70 (t, 1H), 2.7-1.1 (t, 52H), 2.05 (t, 3H), 0.89 (t, 3H), 0.82 (s, 3H).

¹³CNMR (125 MHz, CDCl3, ppm): δ 170.90, 165.36, 151.72, 148.56, 137.07, 136.97, 126.92, 122.43, 122.32, 120.92, 118.73, 82.76, 52.71, 50.98, 46.26, 42.94, 41.40, 38.20, 38.12, 37.06, 34.50, 33.17, 32.50, 32.41, 31.84, 29.85, 29.67, 29.55, 29.49, 29.35, 29.32, 29.18, 29.15, 28.79, 28.16, 26.96, 25.64, 22.78, 22.65, 21.17, 14.63, 12.02.

### Example 9

### Synthesis and Structure Confirmation of Compound IX

0.3 g (0.35 mmol) Compound IV (synthesized in Example 4), 3.5 mL (35 mmol) acetic anhydride and 0.18 g (1.44 mmol) 4-dimethylaminopyridine (DMAP) was sequentially added and then 30 mL tetrahydrofuran was added into a 50 mL round-bottom flask. After reflux reacting for 48 h, the reaction was stopped.

Reaction liquid was treated according to the post process in Example 8 to give milky white colloidal liquid, which was Compound IX.

IR (cm-1): 3311, 2927, 2854, 1736, 1665, 1494, 1460, 1365, 1245, 1200, 1045, 1027, 984, 803, 720.

¹HNMR (500 MHz, CDCl3, ppm): δ 7.27 (t, 1H), 6.82 (t, 1H), 6.76 (t, 1H), 4.70 (t, 1H), 2.77-1.08 (t, 49H), 2.05 (t, 3H), 0.81-0.95 (t, 27H).

¹³CNMR (125 MHz, CDCl3, ppm): δ 174.36, 171.23, 148.55, 136.95, 126.90, 122.45, 122.39, 118.74, 118.71, 82.76, 53.11, 53.03, 52.81, 51.39, 48.45, 48.31, 46.25, 42.94, 41.40, 38.07, 37.78, 37.05, 34.50, 33.16, 32.36, 32.05, 31.93, 31.44, 30.19, 30.05, 30.03, 29.88, 29.67, 29.55, 29.49, 29.35, 29.30, 29.17, 28.80, 28.19, 27.52, 26.99, 25.66, 22.69, 21.17, 20.35, 19.93, 14.63, 12.02.

### Example 10

### Synthesis and Structure Confirmation of Compound X

0.3 g (0.35 mmol) Compound V (synthesized in Example 5), 3.5 mL (35 mmol) butyric anhydride and 0.18 g (1.44 mmol) 4-dimethylaminopyridine (DMAP) was sequentially added and then 30 mL tetrahydrofuran was added into a 50 mL round-bottom flask. After reflux reacting for 48 h, the reaction was stopped.

Reaction liquid was treated according to the post process in Example 8 to give milky white colloidal liquid, which was Compound X.

IR (cm-1): 3441, 2927, 2855, 1734, 1651, 1494, 1460, 1197, 1154, 1120, 1092, 1019, 983, 803, 720.

¹HNMR (500 MHz, CDCl3, ppm): δ 7.27 (t, 1H), 7.15 (t, 1H), 6.88 (t, 1H), 6.81 (d, 1H), 6.40 (t, 1H), 6.00 (d, 1H), 5.63 (t, 1H), 4.70 (t, 1H), 2.7-1.0 (t, 56H), 0.96 (t, 3H), 0.88 (t, 3H), 0.82 (t, 3H).

¹³CNMR (125 MHz, CDCl3, ppm): δ 173.79, 165.38, 151.73, 148.57, 137.02, 136.99, 126.93, 122.45, 122.39, 120.60, 118.67, 82.45, 52.73, 50.99, 46.28, 43.02, 41.42, 38.21, 38.14, 37.10, 36.52, 36.28, 34.51, 33.19, 32.51, 32.43, 31.85, 29.86, 29.68, 29.56, 29.50, 29.36, 29.33, 29.19, 29.17, 28.81, 28.18, 27.58, 26.99, 25.65, 22.82, 22.66, 18.69, 14.50, 12.02, 12.00.

### Example 11

### Synthesis and Structure Confirmation of Compound XI

0.69 g (0.89 mmol) Compound IV (synthesized in Example 4), 14.5 mL (89 mmol) butyric anhydride and 0.44 g (3.52 mmol) 4-dimethylaminopyridine (DMAP) was sequentially added and then 69 mL tetrahydrofuran was added into a 50 mL round-bottom flask. After reflux reacting for 48 h, the reaction was stopped.

After the reaction system was cooled, it was washed with water to neutral and phase separated. The organic layer was spin-dried and purified by silica-gel column chromatography through gradient eluting (the eluent was n-hexane-ethyl acetate (40:1/10:1/5:1, volume ratio)). Then, the eluent was evaporated to dryness to give crude product, which was then treated by ultrasonic water washing for several times. During said water washing process, the crude product attached to walls of flask in the form of colloid and water phase was poured directly after washing. Washing was repeated until the product had no smell of butyric acid. Finally, the product was quickly eluted with ethanol and the solvent was removed in a decompressed oven to give milky white colloidal liquid, which was Compound XI.

IR (cm-1): 3448, 2390, 2857, 1750, 1734, 1609, 1494, 1465, 1364, 1198, 1150, 1121, 1094, 1048, 1019, 984, 905, 803, 732.

¹HNMR (500 MHz, CDCl3, ppm): δ 7.27 (t, 1H), 6.82 (t, 1H), 6.76 (s, 1 H), 4.71 (t, 1H), 1.09-2.77 (t, 53H), 0.81-1.08 (t, 30H).

¹³CNMR (125 MHz, CDCl3, ppm): δ 174.36, 173.78, 148.54, 136.95, 126.90, 122.45, 122.39, 118.73, 118.71, 82.45, 53.33, 53.03, 52.82, 51.39, 48.45, 48.31, 46.27, 43.00, 41.41, 38.08, 37.76, 37.09, 36.52, 34.50, 33.17, 32.36, 32.05, 31.10, 31.07, 30.05, 30.03, 30.01, 29.88, 29.68, 29.59, 29.55, 29.50, 29.35, 29.31, 29.21, 29.17, 28.81, 28.20, 27.57, 27.01, 25.67, 22.58, 22.40, 19.93, 18.59, 14.64, 13.69, 12.06.

### Example of Physicochemical Properties

### Example 12

### Solubility Experiments of Fulvestrant and Ester Derivatives Thereof in Different Solvents

Fulvestrant and ester derivatives of fulvestrant were accurately weighed to an appropriate amount respectively. Their solubilities (in mg/mL) in different oils and solvents were compared according to General Notice in Section 2 of Chinese Pharmacopoeia (2010). The results are shown in Table 1:

TABLE 1
Solubilities of fulvestrant and ester derivatives thereof in
different oils and solvents

| | Castor | Soybean | Medium- | | Propylene |
|---|---|---|---|---|---|
| Solvent | oil | oil | chain oil | PEG 400 | glycol |
| Compound II | >100.2 | >100 | >10 | 2.9 | 10.2 |
| Compound V | ND | 122 | ND | ND | 12.2 |
| Compound I | ND | 255 | ND | ND | 2.9 |
| Compound III | ND | 11 | ND | ND | 0.4 |
| Compound IV | ND | 28 | ND | ND | 7.1 |
| Fulvestrant | 20* | 5 | ND | 6.9 | 10.2 |

Note:
*denote the reported values.

It can be seen that, compared with the solubility of fulvestrant, the solubility of Compound II in lipophilic solvents including castor oil, soybean oil, medium-chain oil increased significantly, yet had almost no change in propylene glycol, and decreased significantly in hydrophilic solvent PEG 400; meanwhile, the solubilities of derivatives such as Compounds I, III and IV in lipophilic soybean oil were significantly greater than that of fulvestrant.

### Example of Drug Efficacy

### Example 13

### The Growth Inhibition Effects of Fulvestrant, Compounds II and X on Human Breast Cancer MCF-7 Xenografted in Nude Mice

Test drugs: fulvestrant, Compounds II and X are dispersed in oil and sterilized to be prepared as oiling agents respectively.

Experimental animals and grouping thereof, source, germline and strain: BALB/c female nude mice, provided by Laboratory Animal Research Center of Academy of Military Medical Sciences of China (Laboratory animal production license: SCXK (Military) 2007-004), day-old: 35-40 days; body weight: 18-24 g. The mice was divided into negative control group, positive control group (fulvestrant oiling agent), drug treatment groups (Compounds II and X oiling agent respectively), with 5 mice in each group.

Administration method, dose and time: the negative control group was administered with blank solvent (oil) by subcutaneous injecting 0.2 mL/20 g for once; positive control group was administered with fulvestrant oiling agent by subcutaneous injecting 100 mg/kg for once; drug treatment groups were respectively administered with Compounds II and X by subcutaneous injecting 100 mg/kg for once.

Establishment of model and tumor measuring method: human breast cancer MCF-7 cell lines in logarithmic growth phase were prepared into a cell suspension of 5×10⁸/mL under sterile condition, with 0.1 mL of which being inoculated to nude mice at their right armpits subcutaneously. Xenografted tumors of nude mice were measured for diameter with vernier caliper, and animals were randomly grouped when the tumors grew to 100-300 mm³. The administration volume to each of the mice was 0.2 mL/20 g by subcutaneous injection at head and neck region. 28 days after administration, the mice were sacrificed and the tumors were stripped by surgery and weighed. Tumor inhibition rate was calculated (inhibition rate=(1-tumor weight in the experimental group/tumor weight in the control group)×100%). The results are shown in Table 2 below:

TABLE 2
The growth inhibition effects of fulvestrant and ester derivatives
thereof on human breast cancer MCF-7 xenografted in nude mice (X ± SD)

| | | | Initial | | Final | | Tumor |
|---|---|---|---|---|---|---|---|
| | Dose | Initial body | animal | Final body | animal | Tumor | inhibition |
| Group | (mg/kg) | weight(g) | number | weight (g) | number | weight (g) | rate (%) |
| Negative | — | 18.800 ± 0.748 | 5 | 21.200 ± 0.748 | 5 | 1.180 ± 0.795 | — |
| control group | | | | | | | |
| Fulvestrant | 100 | 18.400 ± 0.490 | 5 | 15.800 ± 1.166** | 5 | 0.392 ± 0.443 | 66.78 |
| oiling agent | | | | | | | |
| Compound II | 100 | 18.400 ± 0.800 | 5 | 15.200 ± 0.748** | 5 | 0.426 ± 0.306 | 64.90 |
| oiling agent | | | | | | | |
| Compound X | 100 | 18.800 ± 0.748 | 5 | 15.600 ± 1.020** | 5 | 0.402 ± 0.711 | 65.93 |
| oiling agent | | | | | | | |

Compared with the blank control group,
*p < 0.05,
**p < 0.01.

The results show that all of fulvestrant and ester derivatives thereof have anti breast cancer effects.

1. A compound of Formula A: 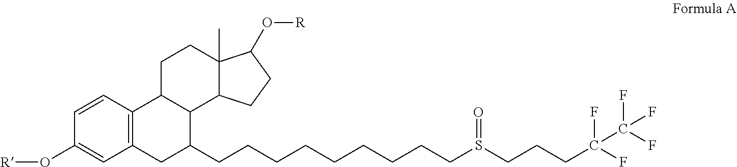
wherein,
substituent R′ is selected from H, alkanoyl or alkenoyl having 2 to 4 carbon atoms,
substituent R is selected from H, alkanoyl or alkenoyl having 2 to 22 carbon atoms.

2. The compound of claim 1, characterized in that,
substituent R′ is H, and substituent R is selected from alkanoyl or alkenoyl having 11 to 22 carbon atoms.

3. The compound of claim 1, characterized in that,
said substituent R is selected from alkanoyl having 11 to 22 carbon atoms, preferably undecanoyl, hexadecanoyl, docosanoyl or 2-[(3′,3′)-dimethyl-1′-methyl]butyl-5-methyl-(7,7)-dimethyl-octanoyl.

4. The compound of claim 1, characterized in that,
said substituent R is selected from alkenoyl containing 1 to 6 carbon-carbon double bonds and having 11 to 22 carbon atoms, wherein said carbon-carbon double bonds can either be distributed in the main chain, or in the branched chain.

5. The compound of claim 13, characterized in that,
said substituent R is selected from undec-2-enoyl, eicosa-5,8,11,14,17-pentaenoyl and docosa-(4,7,10,13,16,19)-hexaenoyl.

6. The compound of claim 1, characterized in that,
when substituent R′ is selected from alkanoyl having 2 to 4 carbon atoms, said alkanoyl is acetyl or butyryl.

7. The compound of claim 1, characterized in that,
said substituent R is selected from alkanoyl or alkenoyl having 11 to 22 carbon atoms, preferably 2-[(3′,3′)-dimethyl-1′-methyl]butyl-5-methyl-(7,7)-dimethyl-octanoyl or undec-2-enoyl.

8. A process for preparing a compound of claim 1, characterized in that, said process comprises the steps of:
a) acylating the —OH at C-17 position of compound of formula B: a compound of formula B is mixed with an alkaline reagent, an organic acid and a catalyst in a solvent at room temperature under stirring to form a reaction mixture, said reaction mixture is reacted to obtain a crude product of compound of Formula A with C-17 position acylated; 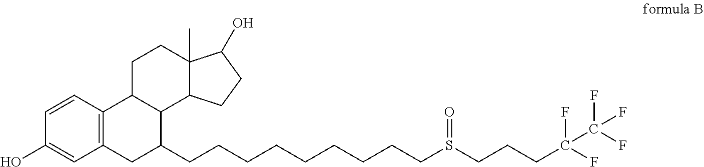 [SMILES: CC12CCC3c4ccc(O)cc4CC(CCCCCCCCCS(=O)CCCC(F)(F)C(F)(F)F)C3C1CCC2O]
b) purifying the crude product obtained in step a) to remove the by-product N,N-dicycloalkylurea and obtain a purified product of compound of Formula A with C-17 position acylated;
when said substituent R′ in the compound is not H, said process further comprises the steps of:
c) acylating C-3 position of the purified product with C-17 position acylated obtained in step b): the purified product with C-17 position acylated obtained in step b) is mixed with an alkaline reagent, an organic acid and a catalyst in a solvent at room temperature under stirring to be reacted to obtain a crude product of compound of Formula A with C-17 and C-3 positions acylated;
d) purifying the crude product obtained in step c) to obtain a purified product of compound of Formula A.

9. The process of claim 8, characterized in that, in step a), said alkaline reagent is selected from pyridine, 2-methylpyridine, 3-methylpyridine, 4-methylpyridine, 2-ethylpyridine, 3-ethylpyridine, 4-ethylpyridine, 5-ethylpyridine, 2-methyl-5-ethylpyridine, 2-dimethylaminopyridine, 4-dimethylaminopyridine, preferably 4-dimethylaminopyridine; said solvent is selected from methyl chloride, methylene chloride, chloroform; said catalyst is dehydrating agent, preferably N,N-dicyclohexylcarbodiimide; said organic acid is alkyl acid or alkenyl acid having 2 to 22 carbon atoms; in step b), said purifying comprises the step of dissolving the crude product obtained in step a) in tetrahydrofuran or ethyl acetate to form a solution, then settling the solution with n-hexane or mixed solvent of n-hexane-ethyl acetate, separating and purifying the settled solution by silica-gel column chromatography and/or neutral alumina adsorption; in step c), said alkaline reagent is selected from pyridine, 2-methylpyridine, 3-methylpyridine, 4-methylpyridine, 2-ethylpyridine, 3-ethylpyridine, 4-ethylpyridine, 5-ethylpyridine, 2-methyl-5-ethylpyridine, 2-dimethylaminopyridine, 4-dimethylaminopyridine, preferably 4-dimethylaminopyridine; said solvent is selected from tetrahydrofuran, ethyl acetate, preferably tetrahydrofuran; said catalyst is dehydrating agent, preferably N,N-dicyclohexylcarbodiimide; said organic acid is alkyl acid or alkenyl acid having 2 to 4 carbon atoms; in step d), said purifying is carried out by silica-gel column chromatography and ethanol elution, wherein mixed solvent of n-hexane-ethyl acetate is used for gradient elution in said silica-gel column chromatography, the volume ratio of n-hexane to ethyl acetate is 50:1-1:1, preferably 40:1/10:1/5:1 for gradient elution.

10. A composition comprising a compound of Formula A of claim 1, characterized in that, said composition is an oiling agent, a fatty agent or a microsphere agent.

11. Use of a composition comprising a compound of Formula A of claim 1 or a composition of claim 10 claim 1 for the manufacture of a medicament in the treatment of cancer; said medicament is preferably used to inhibit cancer cells with estrogen receptors, particularly preferably used to inhibit breast cancer cells.

12. A method for treating cancer, characterized in that, said method comprises administering to a subject in need a therapeutically effective amount of a compound of Formula A of claim 1; said method is preferably used to inhibit cancer cells with estrogen receptors, particularly preferably used to inhibit breast cancer cells;
preferably, said compound of Formula A is administered by injection.

13. The compound of claim 2, characterized in that,
said substituent R is selected from alkanoyl having 11 to 22 carbon atoms, preferably undecanoyl, hexadecanoyl, docosanoyl or 2-[(3′,3′)-dimethyl-1′-methyl]butyl-5-methyl-(7,7)-dimethyl-octanoyl.

14. The compound of claim 2, characterized in that,
said substituent R is selected from alkenoyl containing 1 to 6 carbon-carbon double bonds and having 11 to 22 carbon atoms, wherein said carbon-carbon double bonds can either be distributed in the main chain, or in the branched chain.

15. The compound of claim 13, characterized in that,
said substituent R is selected from undec-2-enoyl, eicosa-5,8,11,14,17-pentaenoyl and docosa-(4,7,10,13,16,19)-hexaenoyl.

16. The compound of claim 6, characterized in that,
said substituent R is selected from alkanoyl or alkenoyl having 11 to 22 carbon atoms, preferably 2-[(3′,3′)-dimethyl-1′-methyl]butyl-5-methyl-(7,7)-dimethyl-octanoyl or undec-2-enoyl.

17. A process for preparing a compound of claim 2, characterized in that, said process comprises the steps of:
a) acylating the —OH at C-17 position of compound of formula B: a compound of formula B is mixed with an alkaline reagent, an organic acid and a catalyst in a solvent at room temperature under stirring to form a reaction mixture, said reaction mixture is reacted to obtain a crude product of compound of Formula A with C-17 position acylated; 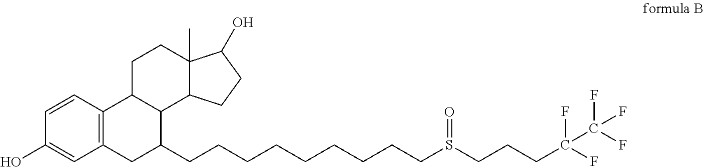 [SMILES: CC12CCC3c4ccc(O)cc4CC(CCCCCCCCCS(=O)CCCC(F)(F)C(F)(F)F)C3C1CCC2O]
b) purifying the crude product obtained in step a) to remove the by-product N,N-dicycloalkylurea and obtain a purified product of compound of Formula A with C-17 position acylated;
when said substituent R′ in the compound is not H, said process further comprises the steps of:
c) acylating C-3 position of the purified product with C-17 position acylated obtained in step b): the purified product with C-17 position acylated obtained in step b) is mixed with an alkaline reagent, an organic acid and a catalyst in a solvent at room temperature under stirring to be reacted to obtain a crude product of compound of Formula A with C-17 and C-3 positions acylated;
d) purifying the crude product obtained in step c) to obtain a purified product of compound of Formula A.

18. A process for preparing a compound of claim 3, characterized in that, said process comprises the steps of:
a) acylating the —OH at C-17 position of compound of formula B: a compound of formula B is mixed with an alkaline reagent, an organic acid and a catalyst in a solvent at room temperature under stirring to form a reaction mixture, said reaction mixture is reacted to obtain a crude product of compound of Formula A with C-17 position acylated; 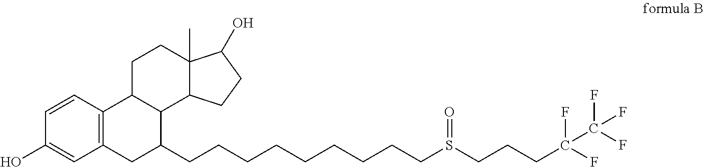 [SMILES: CC12CCC3c4ccc(O)cc4CC(CCCCCCCCCS(=O)CCCC(F)(F)C(F)(F)F)C3C1CCC2O]
b) purifying the crude product obtained in step a) to remove the by-product N,N-dicycloalkylurea and obtain a purified product of compound of Formula A with C-17 position acylated;
when said substituent R′ in the compound is not H, said process further comprises the steps of:
c) acylating C-3 position of the purified product with C-17 position acylated obtained in step b): the purified product with C-17 position acylated obtained in step b) is mixed with an alkaline reagent, an organic acid and a catalyst in a solvent at room temperature under stirring to be reacted to obtain a crude product of compound of Formula A with C-17 and C-3 positions acylated;
d) purifying the crude product obtained in step c) to obtain a purified product of compound of Formula A.

19. A process for preparing a compound of claim 4, characterized in that, said process comprises the steps of:
a) acylating the —OH at C-17 position of compound of formula B: a compound of formula B is mixed with an alkaline reagent, an organic acid and a catalyst in a solvent at room temperature under stirring to form a reaction mixture, said reaction mixture is reacted to obtain a crude product of compound of Formula A with C-17 position acylated; 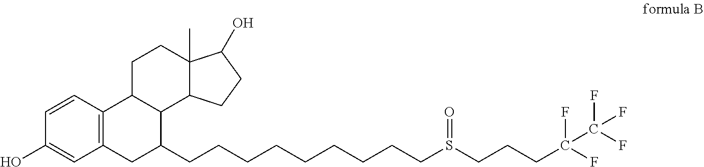 [SMILES: CC12CCC3c4ccc(O)cc4CC(CCCCCCCCCS(=O)CCCC(F)(F)C(F)(F)F)C3C1CCC2O]
b) purifying the crude product obtained in step a) to remove the by-product N,N-dicycloalkylurea and obtain a purified product of compound of Formula A with C-17 position acylated;
when said substituent R′ in the compound is not H, said process further comprises the steps of:
c) acylating C-3 position of the purified product with C-17 position acylated obtained in step b): the purified product with C-17 position acylated obtained in step b) is mixed with an alkaline reagent, an organic acid and a catalyst in a solvent at room temperature under stirring to be reacted to obtain a crude product of compound of Formula A with C-17 and C-3 positions acylated;
d) purifying the crude product obtained in step c) to obtain a purified product of compound of Formula A.

20. The process of claim 17, characterized in that, in step a), said alkaline reagent is selected from pyridine, 2-methylpyridine, 3-methylpyridine, 4-methylpyridine, 2-ethylpyridine, 3-ethylpyridine, 4-ethylpyridine, 5-ethylpyridine, 2-methyl-5-ethylpyridine, 2-dimethylaminopyridine, 4-dimethylaminopyridine, preferably 4-dimethylaminopyridine; said solvent is selected from methyl chloride, methylene chloride, chloroform; said catalyst is dehydrating agent, preferably N,N-dicyclohexylcarbodiimide; said organic acid is alkyl acid or alkenyl acid having 2 to 22 carbon atoms; in step b), said purifying comprises the step of dissolving the crude product obtained in step a) in tetrahydrofuran or ethyl acetate to form a solution, then settling the solution with n-hexane or mixed solvent of n-hexane-ethyl acetate, separating and purifying the settled solution by silica-gel column chromatography and/or neutral alumina adsorption; in step c), said alkaline reagent is selected from pyridine, 2-methylpyridine, 3-methylpyridine, 4-methylpyridine, 2-ethylpyridine, 3-ethylpyridine, 4-ethylpyridine, 5-ethylpyridine, 2-methyl-5-ethylpyridine, 2-dimethylaminopyridine, 4-dimethylaminopyridine, preferably 4-dimethylaminopyridine; said solvent is selected from tetrahydrofuran, ethyl acetate, preferably tetrahydrofuran; said catalyst is dehydrating agent, preferably N,N-dicyclohexylcarbodiimide; said organic acid is alkyl acid or alkenyl acid having 2 to 4 carbon atoms; in step d), said purifying is carried out by silica-gel column chromatography and ethanol elution, wherein mixed solvent of n-hexane-ethyl acetate is used for gradient elution in said silica-gel column chromatography, the volume ratio of n-hexane to ethyl acetate is 50:1-1:1, preferably 40:1/10:1/5:1 for gradient elution.

21. The process of claim 17, characterized in that, in step a), said alkaline reagent is selected from pyridine, 2-methylpyridine, 3-methylpyridine, 4-methylpyridine, 2-ethylpyridine, 3-ethylpyridine, 4-ethylpyridine, 5-ethylpyridine, 2-methyl-5-ethylpyridine, 2-dimethylaminopyridine, 4-dimethylaminopyridine, preferably 4-dimethylaminopyridine; said solvent is selected from methyl chloride, methylene chloride, chloroform; said catalyst is dehydrating agent, preferably N,N-dicyclohexylcarbodiimide; said organic acid is alkyl acid or alkenyl acid having 2 to 22 carbon atoms; in step b), said purifying comprises the step of dissolving the crude product obtained in step a) in tetrahydrofuran or ethyl acetate to form a solution, then settling the solution with n-hexane or mixed solvent of n-hexane-ethyl acetate, separating and purifying the settled solution by silica-gel column chromatography and/or neutral alumina adsorption; in step c), said alkaline reagent is selected from pyridine, 2-methylpyridine, 3-methylpyridine, 4-methylpyridine, 2-ethylpyridine, 3-ethylpyridine, 4-ethylpyridine, 5-ethylpyridine, 2-methyl-5-ethylpyridine, 2-dimethylaminopyridine, 4-dimethylaminopyridine, preferably 4-dimethylaminopyridine; said solvent is selected from tetrahydrofuran, ethyl acetate, preferably tetrahydrofuran; said catalyst is dehydrating agent, preferably N,N-dicyclohexylcarbodiimide; said organic acid is alkyl acid or alkenyl acid having 2 to 4 carbon atoms; in step d), said purifying is carried out by silica-gel column chromatography and ethanol elution, wherein mixed solvent of n-hexane-ethyl acetate is used for gradient elution in said silica-gel column chromatography, the volume ratio of n-hexane to ethyl acetate is 50:1-1:1, preferably 40:1/10:1/5:1 for gradient elution.

22. A composition comprising a compound of Formula A of claim 2, characterized in that, said composition is an oiling agent, a fatty agent or a microsphere agent.

23. A composition comprising a compound of Formula A of claim 3, characterized in that, said composition is an oiling agent, a fatty agent or a microsphere agent.

24. Use of a composition comprising a compound of Formula A of claim 2 for the manufacture of a medicament in the treatment of cancer; said medicament is preferably used to inhibit cancer cells with estrogen receptors, particularly preferably used to inhibit breast cancer cells.

25. Use of a composition comprising a compound of Formula A of claim 3 for the manufacture of a medicament in the treatment of cancer; said medicament is preferably used to inhibit cancer cells with estrogen receptors, particularly preferably used to inhibit breast cancer cells.
